(19) United States
(12) Patent Application Publication
(10) Pub. No.: US 2025/0176544 A1
(43) Pub. Date: Jun. 5, 2025

(54) A process for the Preparation Nitrile Herbicides, their Esters and Salts

(71) Applicant: GHARDA CHEMICALS LIMITED, Thane, Maharashtra (IN)

(72) Inventors: Suchet Saran MATHUR, Thane, Maharashtra (IN); Pragnesh Dalpatram DAMANIA, Thane, Maharashtra (IN); KALIRAJAN A., Thane, Maharashtra (IN); Rahul Harishchandra KHAMKAR, Thane, Maharashtra (IN); Mahesh Vishwas PAWAR, Thane, Maharashtra (IN); Prashant Arun DESHMUKH, Thane, Maharashtra (IN)

(21) Appl. No.: 18/839,284

(22) PCT Filed: Feb. 21, 2023
(86) PCT No.: PCT/IB2023/051557

§ 371 Date: Aug. 16, 2024

(30) Foreign Application Priority Data
Feb. 21, 2022 (IN) 202221009153

(51) Int. Cl. A01N 37/34; A01P 13/00; C07C 253/30

(52) U.S. Cl. CPC A01N 37/34; A01P 13/00; C07C 253/30

(57) ABSTRACT

The present disclosure relates to a process for the preparation of nitrile herbicides, their esters and salts. The process of the present disclosure provides nitrile herbicides, their esters and salts with high purity and in greater yields. Further, the process of the present disclosure is simple, efficient and environment friendly. In addition, the solvent used in the process of the present disclosure can be recycled and reused.

### FIELD

The present disclosure relates to a process for the preparation of nitrile herbicides, their esters and salts. Particularly, the present disclosure relates to a process for the preparation of bromoxynil, its esters and salts.

### BACKGROUND

The background information herein below relates to the present disclosure but is not necessarily prior art.

Herbicides kill or suppress weeds by interfering with its photosynthesis process. The nitrile herbicides, more specifically hydroxybenzonitrile class of herbicide are used as broadleaf weed herbicide, particularly in crop-growing areas.

Various methods for the preparation of the nitrile herbicides are reported in the art. Conventionally, the preparation of the nitrile herbicide is carried out by using organic solvents which are expensive, hazardous to environment and difficult to recover. Moreover, the conventional processes for preparing the nitrile herbicides result in obtaining the product with a low yield and a less purity and thus, not suitable for commercial scale-up. In addition, these conventional processes involve tedious purification stages, thereby making the process expensive.

Therefore, there is felt a need for a process for preparing nitrile herbicides that mitigates the drawbacks mentioned hereinabove or at least provides a useful alternative.

### OBJECTS

Some of the objects of the present disclosure, which at least one embodiment herein satisfies, are as follows:

An object of the present disclosure is to ameliorate one or more problems of the background or to at least provide a useful alternative.

Another object of the present disclosure is to provide a process for the preparation of nitrile herbicides, their esters and salts.

Still another object of the present disclosure is to provide a process for the preparation of nitrile herbicides, their esters and salts with a comparatively high yield and high purity.

Yet another object of the present disclosure is to provide a simple, efficient and economical process for the preparation of nitrile herbicides, their esters and salts.

Still another object of the present disclosure is to provide an environmental friendly process for the preparation of nitrile herbicides, their esters and salts.

Yet another object of the present disclosure is to provide a herbicidal composition comprising nitrile herbicides, their esters and salts.

Other objects and advantages of the present disclosure will be more apparent from the following description, which is not intended to limit the scope of the present disclosure.

### SUMMARY

The present disclosure relates to a process for the preparation of nitrile herbicides, their esters and salts. The process comprises the step of halogenating p-hydroxybenzonitrile by using a halogenating agent (X2) in a first fluid medium at first predetermined conditions to obtain a halogenated compound (nitrile herbicide-Formula II). Separately, an acid (Formula IIIa) is reacted with a chlorinating agent at second predetermined conditions to obtain an acyl chloride (Formula III). The halogenated compound (Formula II) so obtained is esterified with the acyl chloride (Formula III) by using a base in a second fluid medium at third predetermined conditions to obtain the ester of nitrile herbicides (Formula I).

### BRIEF DESCRIPTION OF THE ACCOMPANYING DRAWING

The present disclosure will now be described with the help of the accompanying drawing, in which:

FIG. 1 illustrates a X-ray powder diffractogram of bromoxynil;

FIG. 2 illustrates a X-ray powder diffractogram of bromoxynil heptanoate; and

FIG. 3 illustrates a X-ray powder diffractogram of bromoxynil octanoate.

### DETAILED DESCRIPTION

The present disclosure relates to a process for the preparation of nitrile herbicides, their esters and salts. Particularly, the present disclosure relates to a process for the preparation of bromoxynil, its esters and salts.

Embodiments, of the present disclosure, will now be described with reference to the accompanying drawing.

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, known processes or well-known apparatus or structures, and well known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure are not to be construed as necessarily requiring their performance as described or illustrated. It is also to be understood that additional or alternative steps may be employed.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

Various methods for the preparation of the nitrile herbicides are reported in the art. Conventionally, the preparation of the nitrile herbicide is carried out by using organic solvents which are expensive, hazardous to environment and difficult to recover. Moreover, the conventional processes of preparing the nitrile herbicide results in obtaining the product with a low yield and a less purity and thus, not suitable for commercial scale. In addition, these conventional processes involve tedious purification stages, thereby making the process, expensive.

The present disclosure provides a simple, economic and environmental friendly process for the preparation of nitrile herbicides, their esters and salts and a composition made therefrom.

In an aspect, the present disclosure provides a process for preparing an ester of nitrile herbicides (Formula I).

The process for preparing the ester of nitrile herbicides (Formula I), comprises the following steps:
1. halogenating p-hydroxybenzonitrile by using a halogenating agent (X₂) in a first fluid medium at first predetermined conditions to obtain a halogenated compound (nitrile herbicide-Formula II);
2. separately, reacting an acid (Formula IIIa) with a chlorinating agent at second predetermined conditions to obtain an acyl chloride (Formula III); and
3. esterifying the halogenated compound (Formula II) with the acyl chloride (Formula III) by using a base in a second fluid medium at third predetermined conditions to obtain the ester of nitrile herbicides (Formula I).

The schematic representation of the process for the preparation of the ester of nitrile herbicide (Formula I), is given below as Scheme 1:

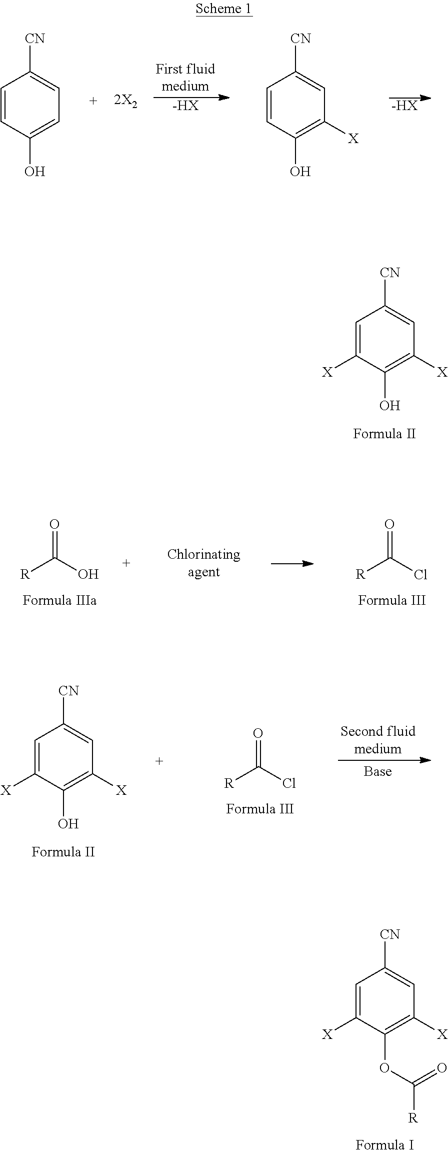

wherein,
R is selected from C₁ to C₁₀ alkyl; and
X is selected from —Br, —Cl and —I.

The process for preparing the ester of nitrile herbicides (Formula I), is described in detail herein below.

### Step 1: Preparation of a Halogenated Compound (Nitrile Herbicide-Formula II)

The process for preparing a halogenated compound (Formula II) comprises the following sub-steps:
a. suspending a predetermined amount of p-hydroxybenzonitrile in a predetermined amount of a first fluid medium to obtain a first mixture;
b. adding a predetermined amount of a halogenating agent (X₂) to the first mixture over a first predetermined time period at a first predetermined temperature to obtain a second mixture;
c. equilibrating the second mixture for a second predetermined time period at a second predetermined temperature to obtain a first product mixture comprising a crude halogenated compound (Formula II); and
d. isolating the crude halogenated compound (Formula II) from the first product mixture and purifying to obtain a pure halogenated compound (Formula II).

The schematic representation of the process for the preparation of the halogenated compound (Formula II), is given below as Scheme 1A:

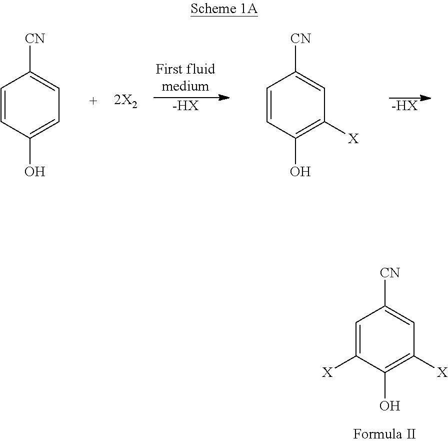

wherein,
X is selected from —Br, —and —I.

The first fluid medium is selected from the group consisting of water, methylene dichloride (MDC), ethylene dichloride (EDC), mono-chloro benzene (MCB) and ortho-dichloro benzen (ODCB). In an exemplary embodiment of the present disclosure, the first fluid medium is water.

The predetermined amount of the first fluid medium is in the range of 0.5 litre to 6 litres per mole of p-hydroxybenzonitrile. In an exemplary embodiment of the present disclosure, the predetermined amount of the first fluid medium is 3 litres per mole of p-hydroxybenzonitrile. In another exemplary embodiment of the present disclosure, the predetermined amount of the first fluid medium is 3.5 litres per mole of p-hydroxybenzonitrile.

In an embodiment of the present disclosure, a molar ratio of p-hydroxybenzonitrile to the halogenating agent (X₂) is in the range of 1:0.5 to 1:3. In an exemplary embodiment of the present disclosure, the molar ratio of p-hydroxybenzonitrile to the halogenating agent (X₂) is 1:2.

Halogenating agent is selected from the group consisting of chlorine gas, liquid bromine and iodine. In an exemplary embodiment of the present disclosure, the halogenating agent is liquid bromine. In another exemplary embodiment of the present disclosure, the halogenating agent is chlorine gas.

The first predetermined time period is in the range of 1 hour to 5 hours depending upon the operating size. In an exemplary embodiment of the present disclosure, the first predetermined time period is 3 hours.

The first predetermined temperature and the second predetermined temperature are independently in the range of 25° C. to 45° C. In an embodiment of the present disclosure, the first predetermined temperature and the second predetermined temperature are in the range of 30° C. to 35° C.

In accordance with an embodiment of the present disclosure, the first predetermined temperature and the second predetermined temperature can be same or different.

The second predetermined time period is in the range of 1 hour to 6 hours. In an exemplary embodiment of the present disclosure, the second predetermined time period is 4 hours.

In an embodiment, the crude halogenated compound (Formula II) is isolated by filtering the first product mixture followed by purifying the crude halogenated compound by washing with water to obtain the pure halogenated compound.

In accordance with an embodiment of the present disclosure, the halogenated compound (Formula II) is a nitrile herbicide.

The nitrile herbicide is selected from the group consisting of bromoxynil, ioxynil and chloroxynil. In an exemplary embodiment of the present disclosure, the halogenated compound (Formula II) is bromoxynil.

The process of preparing the halogenated compound (bromoxynil) in accordance with the present disclosure, involves the use of water as a fluid medium. Thus, the halogenation process of the present disclosure avoids the use of organic solvents that are expensive, hazardous to environment and difficult to recover. Further, the filtrate (water) is recycled and reused, which makes the process environmental friendly. Furthermore, the harmful gases such as HCl and HBr formed during the reaction are not evolved, as the gases (HCl/HBr) get scrubbed in water in the reaction mass and hence, does not pollute air. Moreover, the reaction is carried out at ambient temperatures and hence, no heating or cooling is required, thus the halogenation process of the present disclosure is economical, energy conservative and convenient for a large scale production. The solubility of the bromoxynil in water as well as in water containing HCl/HBr (formed during the halogenation reaction) is very low and hence, the yield and the purity of the product (bromoxynil) is very high.

In an embodiment of the present disclosure, p-hydroxybenzonitrile is prepared by a process comprising the following sub-steps:
a. reacting phenol with bromine in a predetermined ratio in a third fluid medium at a temperature in the range of −10° C. to 0° C. for a time period in the range of 2 hours to 6 hours followed by raising the temperature in the range of 15° C. to 25° C. to obtain a product mixture comprising para-bromophenol, ortho-bromophenol, 2,4-dibromo phenol and 2,4,6-tribromo phenol;
b. subjecting the product mixture to a solvent extraction to obtain a crude mass of para-bromophenol followed by crystallizing the crude mass to obtain a pure para-bromophenol; and
c. reacting para-bromophenol with copper cyanide (CuCN) in a fourth fluid medium at a temperature in the range of 150° C. to 200° C. for a time period in the range of 2 hours to 6 hours to obtain p-hydroxybenzonitrile.

In accordance with the present disclosure, CuBr is formed as a by-product from the reaction of para-bromophenol and CuCN. CuBr is in-situ converted to CuCN back as the starting raw material by using a cheaper reagent aqueous NaCN to avoid any copper carryover.

The predetermined mole ratio of phenol to bromine is in the range of 1:0.5 to 1:2. In an exemplery embodiment of the present disclosure, the predetermined mole ratio of phenol to bromine is 1:1.

The product mixture comprises a mass ratio of para-bromophenol to ortho-bromophenol in the range of 90:10 to 98:2; and a mass ratio of monobromo phenol to dibromo phenol in the range of 95:5 to 99:1. In an exemplery embodiment of the present disclosure, the product mixture comprises the mass ratio of para-bromophenol to ortho-bromophenol as 95:5; and the mass ratio of monobromo phenol to dibromo phenol as 99:1.

The third fluid medium is selected from ethylene dichloride and methylene dichloride. In an exemplery embodiment of the present disclosure, the third fluid medium is ethylene dichloride.

The fourth fluid medium is selected from dimethyl acetamide, dimethyl formamide and N-methylpyrrolidone. In an exemplery embodiment of the present disclosure, the fourth fluid medium is dimethyl acetamide.

The schematic representation of the process for the preparation of p-hydroxybenzonitrile, is given below as Scheme 1A (i):

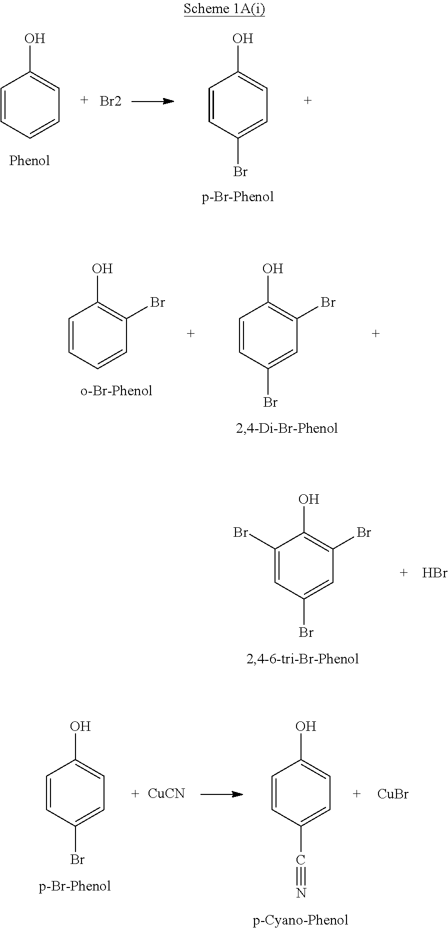

### Step 2: Preparation of an Acyl Chloride (Formula III)

The process for preparing an acyl chloride (Formula III) comprises the following sub-steps:
a. reacting an acid (Formula IIIa) with a chlorinating agent in a predetermined molar ratio at a third predetermined temperature for a third predetermined time period to obtain a second product mixture comprising the acyl chloride (Formula III) and unreacted chlorinating agent; and
b. distilling out the unreacted chlorinating agent from the second product mixture followed by vacuum distillation to obtain the acyl chloride (Formula III).

The schematic representation of the process for the preparation of the acyl chloride (Formula III), is given below as Scheme 1B:

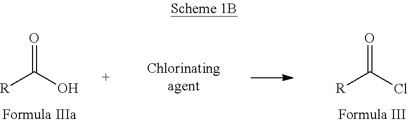

wherein,
R is selected from C₁ to C₁₀ alkyl.

In accordance with an embodiment of the present disclosure, the acid (Formula IIIa) is selected from the group consisting of pentanoic acid, hexanoic acid, heptanoic acid, octanoic acid, nonanoic acid and decanoic acid.

The chlorinating agent is selected from the group consisting of thionyl chloride, phosgene, phosphoryl chloride (POCl₃), phosphorus trichloride (PCl₃) and phosphorus pentachloride (PCl₅).

In an embodiment of the present disclosure, the predetermined molar ratio of the acid (Formula IIIa) to thionyl chloride is in the range of 1:1 to 1:2. In an exemplary embodiment of the present disclosure, the molar ratio of the acid (Formula IIIa) to thionyl chloride is 1:1.25.

The third predetermined temperature is in the range of 30° C. to 75° C. In an embodiment of the present disclosure, the third predetermined temperature is in the range of 55° C. to 65° C.

The third predetermined time period is in the range of 2 hours to 10 hours. In an exemplary embodiment of the present disclosure, the third predetermined time period is 4 hours.

In accordance with an embodiment of the present disclosure, the acyl chloride (Formula III) is selected from the group consisting of pentanoyl chloride, hexanoyl chloride, heptanoyl chloride, octanoyl chloride, nonanoyl chloride and decanoyl chloride.

In an embodiment of the present disclosure, the recovered unreacted thionyl chloride is recycled and reused in the next batch, thereby making the process of the present disclosure cost efficient, economic and environmental friendly.

### Step 3: Preparation of the Ester of Nitrile Herbicide (Formula I)

The process for preparing the ester of nitrile herbicide (Formula I), comprises the following sub-steps:
a. mixing a predetermined amount of the halogenated compound (nitrile herbicide-Formula II) prepared in step-1 and a base in a predetermined amount in a second fluid medium to obtain a mixture;
b. adding a predetermined amount of the acyl chloride (Formula III) prepared in step-2 to the mixture over a fourth predetermined time period at a fourth predetermined temperature to obtain a reaction mass;
c. equilibrating the reaction mass for a fifth predetermined time period at a fifth predetermined temperature to obtain a third product mixture comprising a crude ester of nitrile herbicide (Formula I); and
d. isolating the crude ester of nitrile herbicide (Formula I) from the third product mixture and purifying to obtain the pure ester of nitrile herbicide (Formula I).

The schematic representation of the process for the preparation of the ester of nitrile herbicides (Formula I), is given below as Scheme 1C:

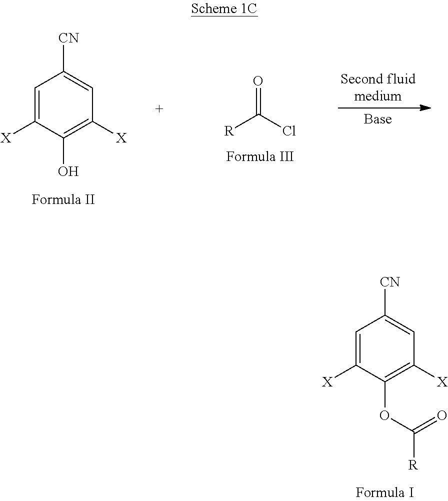

wherein,
R is selected from C₁ to C₁₀ alkyl; and
X is selected from —Br, —Cl and —I.

In accordance with the present disclosure, the base is selected from an inorganic base and an organic base.

The organic base is selected from the group consisting of trimethylamine, triethylamine, tributylamine, alkylpyridine, quinoline and a combination thereof. In an exemplary embodiment of the present disclosure, the organic base is triethylamine.

In an embodiment of the present disclosure, the alkylpridine is selected from 2,6-lutidine and 3,5-lutidine.

The inorganic base is selected from the group consisting of sodium carbonate, sodium bicarbonate, potassium carbonate, potassium bicarbonate, sodium hydroxide, potassium hydroxide and a combination thereof.

In an embodiment of the present disclosure, a molar ratio of the halogenated compound (nitrile herbicide-Formula II) to the base is in the range of 1:1 to 1:3. In an exemplary embodiment of the present disclosure, the molar ratio of the halogenated compound (Formula II) to the base is 1:1.2.

The second fluid medium is selected from the group consisting of toluene, acetonitrile, monochlorobenzene, methylene dichloride, ethylene dichloride, o-dichlorobenzene, xylene and methyl ethyl ketone. In an exemplary embodiment of the present disclosure, the second fluid medium is toluene.

In an embodiment of the present disclosure, a molar ratio of the halogenated compound (Formula II) to the acyl chloride (Formula III) is in the range of 1:0.9 to 1:1.1. In an exemplary embodiment of the present disclosure, the molar ratio of the halogenated compound (Formula II) to the acyl chloride (Formula III) is 1:1.05.

The fourth predetermined temperature and the fifth predetermined temperature are independently in the range of 20° C. to 40° C. In an embodiment of the present disclosure, the fourth predetermined temperature and the fifth predetermined temperature are independently in the range of 25° C. to 30° C.

In accordance with an embodiment of the present disclosure, the fourth predetermined temperature and the fifth predetermined temperature can be same or different.

The fourth predetermined time period is in the range of 0.5 hour to 5 hours. In an exemplary embodiment of the present disclosure, the fourth predetermined time period is 2 hours.

The fifth predetermined time period is in the range of 1 hour to 10 hours. In an exemplary embodiment of the present disclosure, the fifth predetermined time period is 6 hours.

In accordance with an embodiment of the present disclosure, the ester of nitrile herbicide (Formula I) is selected from the group consisting of bromoxynil pentanoate, bromoxynil hexanoate, bromoxynil heptanoate, bromoxynil octanoate, bromoxynil nonanoate and bromoxynil decanoate.

The present disclosure provides an alternative process for the preparation of esters of nitrile herbicides (bromoxynil esters) by using non-toxic and cheap reagents. As a result of using non-toxic, inexpensive and easily available reagents, the process of the present disclosure is cost efficient, economic and environmental friendly.

In another aspect, the present disclosure envisages a herbicidal composition.

In an embodiment of the present disclosure, the herbicidal composition comprises at least one nitrile herbicide (Formula II), at least one ester of nitrile herbicide (Formula I) and salts thereof.

In an exemplary embodiment of the present disclosure, the herbicidal composition comprises bromoxynil heptanoate and bromoxynil octanoate in a predetermined mass ratio.

In accordance with the embodiment of the present disclosure, the predetermined mass ratio is in the range of 1:99 to 99:1.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further illustrated herein below with the help of the following experiments. The experiments used herein are intended merely to facilitate an understanding of the ways in which the embodiments herein may be practiced and to further enable those of skill in the art to practice the embodiments herein. Accordingly, the experiments should not be construed as limiting the scope of embodiments herein. These laboratory scale experiments can be scaled up to industrial/commercial scale and the results obtained can be extrapolated to industrial/commercial scale.

### EXPERIMENTAL DETAILS

Experiment 1: Preparation of Bromoxynil Esters in Accordance with the Present Disclosure

### Step 1: Preparation of a Halogenated Compound (Formula II)

I. Preparation of p-Hydroxybenzonitrile:
Sub-Step-1 Preparation of p-Bromophenol

Example (i): 94.11 g of phenol (1 mole) was taken in 200 ml ethylene dichloride (EDC) and cooled to −5° C. to obtain a phenol solution. 160 g of bromine (1 mole) was diluted with 100 ml EDC and added slowly to the phenol solution over 6 hours to obtain a mass. The mass was stirred at −5° C. for next 4 hours and then the temperature was raised to 20° C. to obtain a product mixture comprising p-bromophenol and o-bromophenol in a mass ratio of 95:5 and mono-bromo phenol to dibromo-phenol in a mass ratio of 99:1. The progress of the reaction was monitored by GC analysis. GC analysis showed <1% unreacted starting phenol.

100 ml water was added to the product mixture at <10° C. and washed the EDC layer with aqueous Na₂SO₂O₃, aqueous Na₂CO₃ and neutral water to obtain a washed EDC layer. The washed EDC layer was concentrated to obtain 166 g of product corresponding to 95 wt % crude p-bromophenol.

Crude p-bromophenol was crystallized by using 0.5 ml/gm hexane to get 85% useable p-bromophenol of >99% GC area purity. Over all yield was 81 mole % with respect to the starting material phenol used.

Comparative example (i): 23.6 g phenol (0.25 mole) was taken in 200 ml/mole EDC at 30° C. to obtain a phenol solution. 40 g bromine (1 mole) was diluted with 100 ml/mole EDC and added slowly to the phenol solution over 4 hours to obtain a mass. The mass was stirred at 30° C. for next 4 hours to obtain a product mixture comprising p-bromophenol and o-bromophenol in a mass ratio of 75:25 and mono-bromo phenol to dibromo-phenol in a mass ratio of 95:5. The progress of the reaction was monitored by GC analysis. GC analysis showed 3% unreacted starting phenol. Reaction was worked up by following the same procedure as in example (i).

From Examples (i) and (ii), it was observed that the lower temperature during bromination results in higher yield of para-bromophenol.

Comparative example (iii): 23.6 g phenol (0.25 mole) was taken in 300 ml/mole water at −5 to 0° C. to obtain a phenol solution. 40 g bromine (1 mole) was added to the phenol, solution over 2 hours to obtain a mass. The mass was stirred at −5 to 0° C. for next 4 hours to obtain a product mixture comprising p-bromophenol and o-bromophenol in a mass ration of 87:13 and mono-bromophenol to dibromo-phenol in a mass ratio of 64:26. The progress of the reaction was monitored by GC analysis. GC analysis showed 20% unreacted starting phenol. Reaction was worked up by following the same procedure as in example (i).

Thus, phenol bromination in water gave higher dibromo-phenol content.

Sub-Step-2 Preparation of p-Hydroxy Benzonitrile (Cyanation Using CuCN)

Example (i): 173 g p-bromophenol (1 mole) so obtained in sub-step-1 was taken in 350 ml dimethyl acetamide (DMAC) to obtain a solution of p-bromophenol. 117 g of CuCN solid (1.3 moles) was added to the solution under high purity nitrogen atmosphere to obtain a reaction mass. The reaction mass was heated to 180° C. and maintained for 4 hours to obtain a product mass.

The DMAC solvent (300 ml) was then distilled out from the product mass under vacuum to obtain a residual mass. 50 g of NaCN (1 mole) dissolved in water (265 ml) was added to the residual mass and stirred for 1 hour at 100° C. followed by cooling the contents to 30° C. and filtered to obtain a filtrate (comprising aqueous NaBr and product) and a solid cake. The solid cake was then treated with methanol to obtain a methanol washing and a solid part which was recycled in the next batch as CuCN.

CuBr was formed as a by-product from the reaction of para-bromophenol and CuCN. CuBr reacted with aqueous NaCN and got converted to CuCN and NaBr. NaBr was removed as a by-product and CuCN was recycled as the starting material.

The filtrate was separated from the aqueous NaBr solution to obtain an organic phase containing product which was mixed with the methanol washings. The organic phase and methanol washings were combined and concentrated to obtain the product mass and to recover solvent (methanol and remaining DMAC). The recovered solvent was recycled in the next batch. The product mass was vacuum distilled and used in the next step. The yield of the distilled p-hydroxybenzonitrile was 80%. The product had <10 ppm cyanide content. Recovered CuCN purity by copper was 96%.

Examples (ii) to (v): p-hydroxybenzonitrile was prepared by using the same procedure of Example (i) by varying the reaction temperature and time period to study the % conversion of p-bromophenol to phydroxybenzonitrile. The results are summarized in table 1 below.

TABLE 1

| | Reaction | Time period in | Conversion by GC |
|---|---|---|---|
| Example no | Temperature | hours | analysis |
| ii | 125° C. | 24 | 2% |
| iii | 135° C. | 10 | 87% |
| iv | 150° C. | 12 | 98% |
| v | 180° C. | 4 | 98% |

From Table 1, it is observed that a minimum temperature is required to shift the equilibrium to right side (product formation).

Examples (vi) to (x): The solids obtained from example (i) as CuCN was employed in the reaction of Example (vi) instead of fresh CuCN and the process was performed as per example (i). Again the solid CuCN obtained in Example (vi) was isolated and similarly CuCN was recycled in next four batches. The results are summarized in Table 2 below.

TABLE 2

| | | Conversion by GC | |
|---|---|---|---|
| Example no | CuCN source | analysis | Yield |
| vi | Fresh | 95% | 80% |
| vii | 1^{st} recycle | 95% | 75% |
| viii | 2^{nd} recycle | 95% | 80% |
| ix | 3^{rd} recycle | 87% | 87% |
| x | 4^{th} recycle | 92% | 87% |

From Table 2, it is observed that recycle of recovered CuCN is established after using it, instead of fresh CuCN. Thus economically viable.

### II. Preparation of Halogenated Compound:

### Example 1a: Preparation of 3,5-dibromo-4-hydroxybenzonitrile (Bromoxynil)

In a glass reactor equipped with a stirrer, 715 g of p-hydroxybenzonitrile (6 moles) was charged and 21 litres of water was added to obtain a first mixture. 2008 g of liquid bromine (12.3 moles) was slowly added to the first mixture over 3 hours at 30° C. to obtain a second mixture. The second mixture was equilibrated for 4 hours at 35° C. to obtain a first product mixture comprising a crude 3,5-dibromo-4-hydroxy benzonitrile. The crude 3,5-dibromo-4-hydroxybenzonitrile was isolated by filtering the first product mixture to obtain a filtrate and a residue containing crude 3,5-dibromo-4-hydroxybenzonitrile followed by purifying the crude 3,5-dibromo-4-hydroxybenzonitrile by washing the with water and drying to obtain the pure 3,5-dibromo-4-hydroxybenzonitrile having 99.0% purity and 97.7 wt % yield. The water filtrate so obtained can be recycled and reused in the next batch.

### Example 1b: Preparation of 3,5-dibromo-4-hydroxybenzonitrile (Bromoxynil)

The same procedure Example 1a was repeated except the water filtrate (6750 ml) so obtained in Example 1 was used instead of fresh fluid medium (water) and 268 g of p-hydroxybenzonitrile (2.25 moles) and 742 g of liquid bromine (4.55 moles) were used to obtain 3,5-dibromo-4-hydroxybenzonitrile having 97.7% purity and 98.0 wt % yield.

Further, the water filtrate of previous batches containing HBr was recycled and reused in the next batches for four more times.

### Example 1c: Preparation of 3-chloro-5-bromo-4-hydroxybenzonitrile

In a glass reactor equipped with a stirrer, 5 g of 3-chloro-4-hydroxybenzonitrile (0.0325 mole) was charged and 0.1 litre of demineralized water was added to obtain a first mixture. 4.7 g of liquid bromine (0.029 mole) was slowly added to the first mixture over 1 hour at 30° C. to obtain a second mixture. The second mixture was equilibrated for 1 hour at 30° C. to obtain a first product mixture comprising a crude 3-chloro-5-bromo-4-hydroxybenzonitrile. The crude 3-chloro-5-bromo-4-hydroxybenzonitrile was isolated by filtering the first product mixture to obtain a filtrate and a residue containing 3-chloro-5-bromo-4-hydroxybenzonitrile followed by purifying the crude 3-chloro-5-bromo-4-hydroxybenzonitrile by washing with water and drying to obtain the pure 3-chloro-5-bromo-4-hydroxybenzonitrile having 89.7% purity and 89 wt % yield.

### Example 1d: Preparation of 3,5-dichloro-4-hydroxybenzonitrile

In a glass reactor equipped with a stirrer, 59.5 g of 4-hydroxybenzonitrile (0.5 mole) was charged and 1.3 litres of demineralized water was added to obtain a first mixture. Chlorine gas was bubbled through the first mixture over 3 hours at 30° C. to obtain a second mixture. The second mixture was equilibrated for 4 hours at 30° C. to obtain a first product mixture comprising a crude 3,5-dichloro-4-hydroxy benzonitrile. The crude 3,5-dichloro-4-hydroxybenzonitrile was isolated by filtering the first product mixture to obtain a filtrate and a residue containing 3,5-dichloro-4-hydroxybenzonitrile followed by purifying the crude 3,5-dichloro-4-hydroxybenzonitrile by washing with water and drying to obtain the pure 3,5-dichloro-4-hydroxybenzonitrile having 80.7% purity and 60 wt % yield.

### STEP 2: Preparation of an Acyl Chloride (Formula III)

### Example 2a: Preparation of Heptanoyl Chloride

In a reactor, 1 mole of heptanoic acid (130 g) was reacted with 1.25 moles of thionyl chloride (149 g) by heating at 60° C. and further maintaining at 60° C. for 4 hours to obtain a second product mixture comprising the heptanoyl chloride and unreacted thionyl chloride. The unreacted thionyl chloride was distilled out from the second product mixture followed by vacuum distilling the second product mixture to obtain the heptanoyl chloride having 98% purity and 95 mole % yield.

The distilled thionyl chloride was recycled and reused in the next batch.

### Example 2b: Preparation of Octanoyl Chloride

The same procedure Example 2a was repeated except the octanoic acid was used instead of heptanoic acid to obtain octanoyl chloride.

Similarly, pentanoyl chloride, hexanoyl chloride, nonanoyl chloride and decanoyl chloride was prepared by using pentanoic acid, hexanoic acid, nonanoic acid and decanoic acid respectively (by using the same process conditions as stated in Example 2a).

### Step 3: Preparation of Bromoxynil Ester

### Example 3a: Preparation of Bromoxynil Heptanoate

2 moles of 3,5-Dibromo-4-hydroxybenzonitrile (bromoxynil) prepared in Example 1a of step-1 was mixed with 1 litre of toluene and 2.2 moles of triethylamine (223 g) to obtain a mixture. 2.1 moles of heptanoly chloride (312 g) prepared in Example 2a of step-2 was added to the mixture over 2 hours at 35° C. to obtain a reaction mass. The reaction mass was equlibrated for 6 hours at 35° C. to obtain a third product mixture comprising a crude bromoxynil heptanoate. The crude bromoxynil heptanoate (763 g) was isolated by workup and toluene concentration and then purified with n-hexane to obtain a pure bromoxynil heptanoate having 99.5% purity and 88 wt % yield.

### Example 3b: Preparation of Bromoxynil Octanoate

1.885 moles of 3,5-Dibromo-4-hydroxybenzonitrile (bromoxynil) prepared in Example 1a of step-1 was mixed with 0.945 litre of toluene and 2.074 moles of triethylamine (TEA) (210 g) to obtain a mixture. 1.98 moles of heptanoly chloride (322 g) prepared in Example 2b of step-2 was added to the mixture over 2 hours at 35° C. to obtain a reaction mass. The reaction mass was equlibrated for 4 hours at 35° C. to obtain a third product mixture comprising a crude bromoxynil octanoate. The crude bromoxynil octanoate (751.4 g) was isolated by workup and toluene concentration and then purified with n-hexane to obtain a pure bromoxynil octanoate having 99.5% purity and 88 wt % yield.

Similarly, the same procedure as in Example 3a was followed to prepare bromoxynil pentanoate, bromoxynil hexanoate, bromoxynil nonanoate, bromoxynil decanoate were prepared by using pentanoyl chloride, hexanoyl chloride, nonanoyl chloride and decanoyl chloride respectively.

Experiment 2: Preparation of Salts of Bromoxynil in Accordance with the Present Disclosure

### Example (a): Preparation of Potassium Salt of Bromoxynil

1 g (0. 00361 mole) of 3,5-Dibromo-4-hydroxybenzonitrile (bromoxynil) prepared in Example 1a of step-1 was taken in 20 ml of water to obtain a reaction mixture. 1.05 mole of KOH was added to the mixture at 40° C. and was stirred for 1 hour to obtain a clear solution comprising potassium salt of bromoxynil.

### Example (b): Preparation of Sodium Salt of Bromoxynil

Sodium salt of bromoxynil is prepared by following the same procedure as in Example (a) except that NaOH is used instead of KOH

### Example (c): Preparation of Triethylamine Salt of Bromoxynil

Triethylamine salt of bromoxynil is prepared by following the same procedure as in Example (a) except that triethylamine is used instead of KOH.

### Experiment 3: Characterization of Bromoxynil, Bromoxynil Heptanoate and Bromoxynil Octanoate

The bromoxynil (Example 1a), bromoxynil heptanoate (Example 3a) and bromoxynil octanoate (Example 3b) prepared in Experiment 1 were characterized by X-ray powder diffraction and the characteristic reflexes as 2θ+0.2 degree in the X-ray powder diffractogram recorded using Cu—Kα radiation at 25° C. are summarized in Table 3.

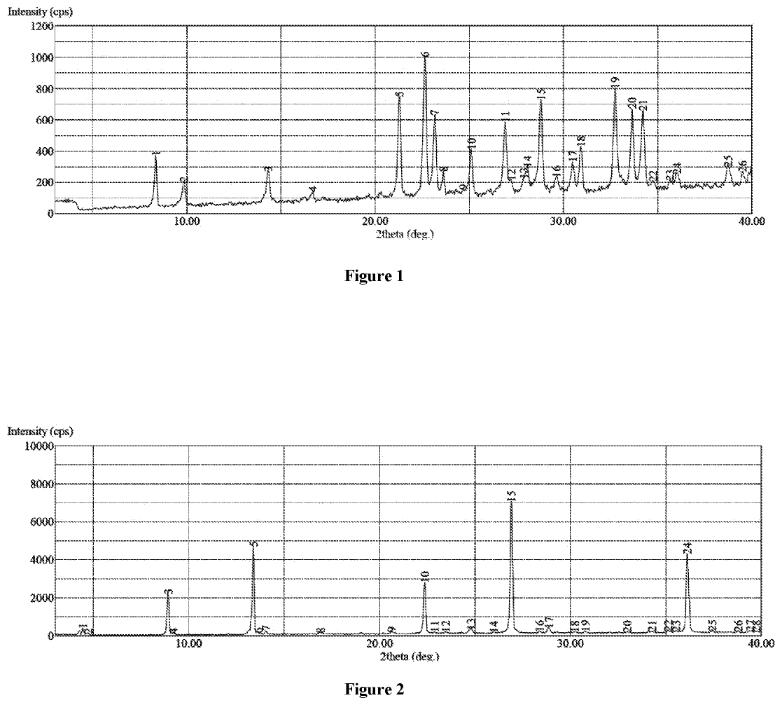

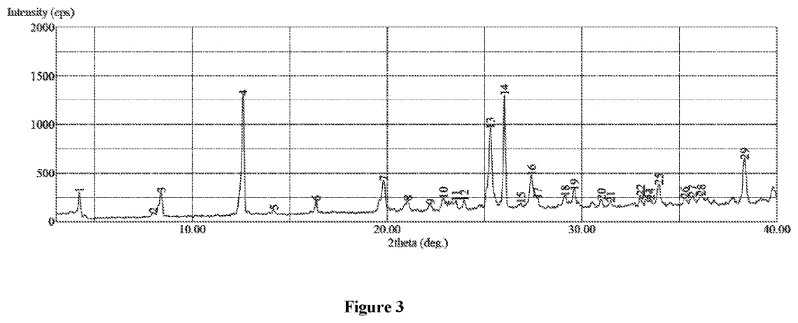

The X-ray powder diffractograms of bromoxynil, bromoxynil heptanoate and bromoxynil octanoate are illustrated in FIGS. 1 to 3 respectively.

TABLE 3
2θ ± 0.2 degree values of bromoxynil, bromoxynil
heptanoate and bromoxynil octanoate

| | | 2θ ± 0.2 degree | 2θ ± 0.2 degree |
|---|---|---|---|
| | 2θ ± 0.2 degree | of bromoxynil | of bromoxynil |
| Sr. No. | of bromoxynil | heptanoate | octanoate |
| 1. | 8.340 | 8.920 | 8.400 |
| 2. | 9.840 | 13.380 | 12.580 |
| 3. | 21.280 | 14.040 | 19.780 |
| 4. | 22.640 | 22.360 | 22.860 |
| 5. | 23.160 | 22.880 | 25.300 |
| 6. | 28.800 | 24.780 | 25.960 |
| 7. | 32.740 | 26.900 | 27.400 |
| 8. | 34.220 | — | 29.580 |

The 2θ values confirms the formation of bromoxynil, bromoxynil heptanoate and bromoxynil octanoate.

Experiment 4: Preparation of the Herbicidal Composition in Accordance with the Present Disclosure

### Example 4a: Preparation of the Herbicidal Composition by Reacting Bromoxynil with a Mixture of Heptanoyl Chloride and Octanoyl Chloride

0.2 mole of 3,5-Dibromo-4-hydroxybenzonitrile (bromoxynil) (55.5 g) was mixed with 0.2 litre of toluene and 0.24 moles TEA (25 g) to obtain a mixture. In another flask, 0.11 mole of octanoyl chloride (18 g) and 0.11 mole of heptanoyl chloride (16.5 g) were mixed well for 30 minutes to obtain an acyl chloride mixture. This mixture of octanoyl chloride and heptanoyl chloride was added to the mixture at 35° C. over 2 hours to obtain a reaction mass. The reaction mass was equlibrated for 6 hours at 35° C. to obtain a product mixture comprising a crude product mixture of bromoxynil heptanoate and bromoxynil octanoate. The crude product mixture (62.6 g) of bromoxynil heptanoate and bromoxynil octanoate was isolated by workup and toluene concentration and then purified with n-hexane to obtain a pure product containing 48.5% bromoxynil heptanoate and 50.78% bromoxynil octanoate.

### Example 4b: Preparation of the Herbicidal Composition by Mixing of Bromoxynil Heptanoate and Bromoxynil Octanoate in a Desired Weight Ratio

101.18 g of bromoxynil heptanoate was mixed with 105.14 g of bromoxynil octanoate in a moisture free environment and stirred at 50° C. for 2 hours to obtain a clear solution. On analysis (GC analysis), it was found that the composition comprised 47.9% bromoxynil heptanoate and 50.6% bromoxynil octanoate.

### Technical Advances and Economical Significance

The present disclosure described herein above has several technical advantages including, but not limited to, the realization of a process for the preparation of nitrile herbicides, their esters and salts, wherein:
the process provides nitrile herbicides, their esters and salts with high purity and in greater yields;
the process is simple, efficient and environment friendly; and
the solvent can be recycled and reused.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

The invention claimed is:

1. A process for preparing an ester of nitrile herbicide of Formula I, 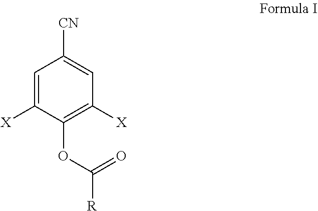
said process comprising the following steps:
1. halogenating p-hydroxybenzonitrile by using a halogenating agent (X₂) in a first fluid medium at first predetermined conditions to obtain a halogenated compound (nitrile herbicide-Formula II); 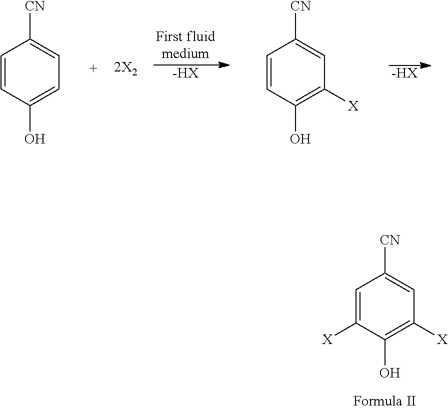
wherein,
X is selected from —Br, —and —I,
2. separately, reacting an acid (Formula IIIa) with a chlorinating agent at second predetermined conditions to obtain an acyl chloride (Formula III); and 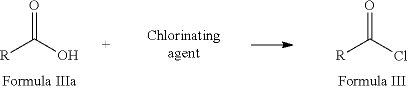
wherein,
R is selected from C₁ to C₁₀ alkyl,
3. esterifying said halogenated compound (Formula II) with said acyl chloride (Formula III) by using a base in a second fluid medium at third predetermined conditions to obtain said ester of nitrile herbicide (Formula I). 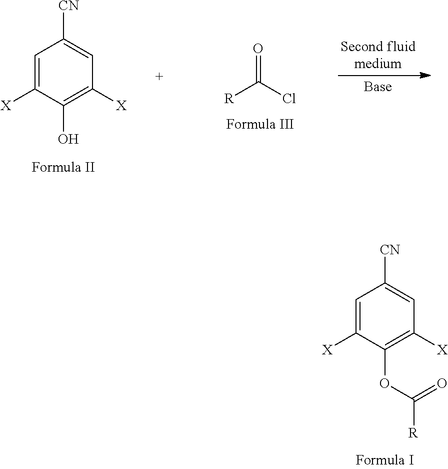
wherein,
R is selected from C₁ to C₁₀ alkyl; and
X is selected from —Br, —Cl and —I.

2. The process as claimed in claim 1, wherein said p-hydroxybenzonitrile is prepared by a process comprising the following sub-steps:
a. reacting phenol with bromine in a predetermined mole ratio in a third fluid medium at a temperature in the range of −10° C. to 0° C. for a time period in the range of 2 hours to 6 hours followed by raising the temperature in the range of 15° C. to 25° C. to obtain a product mixture comprising para-bromophenol, ortho-bromophenol, 2,4-dibromo phenol and 2,4,6-tribromo phenol;
b. subjecting said product mixture to a solvent extraction to obtain a crude mass of para-bromophenol followed by crystallizing said crude mass to obtain a pure para-bromophenol; and
c. reacting said pure para-bromophenol with copper cyanide (CuCN) in a fourth fluid medium at a temperature in the range of 150° C. to 200° C. for a time period in the range of 2 hours to 6 hours to obtain p-hydroxybenzonitrile.

3. The process as claimed in claim 2, wherein
said predetermined mole ratio of said phenol to said bromine is in the range of 1:0.5 to 1:2; and
said product mixture comprises
a mass ratio of said para-bromophenol to said ortho-bromophenol in the range of 90:10 to 98:2; and
a mass ratio of said monobromo phenol to said dibromo phenol in the range of 95:5 to 99:1.

4. (canceled)

5. The process as claimed in claim 2, wherein said third fluid medium is selected from ethylene dichloride and methylene dichloride; and said fourth fluid medium is selected from dimethyl acetamide, dimethyl formamide and N-methylpyrrolidone.

6. The process as claimed in claim 1, wherein said step (1) of preparing said halogenated compound (Formula II) comprises the following sub-steps:
a. suspending a predetermined amount of p-hydroxybenzonitrile in a predetermined amount of a first fluid medium to obtain a first mixture;
b. adding a predetermined amount of a halogenating agent (X₂) to said first mixture over a first predetermined time period at a first predetermined temperature to obtain a second mixture;
c. equilibrating said second mixture for a second predetermined time period at a second predetermined temperature to obtain a first product mixture comprising a crude halogenated compound (Formula II); and
d. isolating said crude halogenated compound (Formula II) from said first product mixture and purifying to obtain a pure halogenated compound (Formula II).

7. The process as claimed in claim 5, wherein said first fluid medium is selected from the group consisting of water, methylene dichloride (MDC), ethylene dichloride (EDC), mon-chloro benzene (MCB) and ortho-dichloro benzen (ODCB); and wherein said predetermined amount of said first fluid medium is in the range of 0.5 litre to 6 litres per mole of p-hydroxybenzonitrile.

8. The process as claimed in claim 5, wherein
a molar ratio of p-hydroxybenzonitrile to said halogenating agent (X₂) is in the range of 1:0.5 to 1:3;
said first predetermined time period is in the range of 1 hour to 5 hours;
said second predetermined time period is in the range of 1 hour to 6 hours; and
said first predetermined temperature and said second predetermined temperature are independently in the range of 25° C. to 45° C.

9. The process as claimed in claim 5, wherein said halogenating agent is selected from the group consisting of chlorine gas, liquid bromine and iodine; and said halogenated compound (Formula II) is a nitrile herbicide; and wherein said nitrile herbicide is selected from the group consisting of bromoxynil, ioxynil and chloroxynil.

10. (canceled)

11. (canceled)

12. The process as claimed in claim 1, wherein said step (2) of preparing said acyl chloride (Formula III) comprises the following sub-steps:
a. reacting said acid (Formula IIIa) with a chlorinating agent in a predetermined molar ratio at a third predetermined temperature for a third predetermined time period to obtain a second product mixture comprising the acyl chloride (Formula III) and unreacted chlorinating agent; and
b. distilling out said unreacted chlorinating agent from said second product mixture followed by vacuum distillation to obtain said acyl chloride (Formula III).

13. The process as claimed in claim 9, wherein said acid (Formula IIIa) is selected from the group consisting of pentanoic acid, hexanoic acid, heptanoic acid, octanoic acid, nonanoic acid and decanoic acid.

14. The process as claimed in claim 9, wherein said chlorinating agent is selected from the group consisting of thionyl chloride, phosgene, phosphoryl chloride (POCl₃), phosphorus trichloride (PCl₃) and phosphorus pentachloride (PCl₅).

15. The process as claimed in claim 9, wherein said predetermined molar ratio of said acid (Formula IIIa) to thionyl chloride is in the range of 1:1 to 1:2; said third predetermined temperature is in the range of 30° C. to 75° C.; and said third predetermined time period is in the range of 2 hours to 10 hours.

16. (canceled)

17. The process as claimed in claim 9, wherein said acyl chloride (Formula III) is selected from the group consisting of pentanoyl chloride, hexanoyl chloride, heptanoyl chloride, octanoyl chloride, nonanoyl chloride and decanoyl chloride.

18. The process as claimed in claim 1, wherein said step (3) of preparing said ester of nitrile herbicide (Formula I), comprises the following sub-steps:
a. mixing a predetermined amount of said halogenated compound (nitrile herbicide-Formula II) prepared in step (i) and a base in a predetermined amount in a second fluid medium to obtain a mixture;
b. adding a predetermined amount of said acyl chloride (Formula III) prepared in step (ii) to said mixture over a fourth predetermined time period at a fourth predetermined temperature to obtain a reaction mass;
c. equilibrating said reaction mass for a fifth predetermined time period at a fifth predetermined temperature to obtain a third product mixture comprising a crude ester of nitrile herbicide (Formula I); and
d. isolating said crude ester of nitrile herbicide (Formula I) from said third product mixture and purifying to obtain said pure ester of nitrile herbicide (Formula I).

19. The process as claimed in claim 14, wherein said base is selected from an inorganic base and an organic base; wherein said organic base is selected from the group consisting of trimethylamine, triethylamine, tributylamine alkylpyridine, quinoline and a combination thereof; and said inorganic base is selected from the group consisting of sodium carbonate, sodium bicarbonate, potassium carbonate, potassium bicarbonate, sodium hydroxide, potassium hydroxide and a combination thereof.

20. (canceled)

21. The process as claimed in claim 14, wherein
a. a molar ratio of said halogenated compound (nitrile herbicide-Formula II) to said base is in the range of 1:1 to 1:3;
b. a molar ratio of said halogenated compound (Formula II) to said acyl chloride (Formula III) is in the range of 1:0.9 to 1:1.1;
c. said fourth predetermined temperature and said fifth predetermined temperature are independently in the range of 20° C. to 40° C.;
d. said fourth predetermined time period is in the range of 0.5 hour to 5 hours; and
e. said fifth predetermined time period is in the range of 1 hour to 10 hours.

22. The process as claimed in claim 14, wherein said second fluid medium is selected from the group consisting of toluene, acetonitrile, monochlorobenzene, methylene dichloride, ethylene dichloride, o-dichlorobenzene, xylene and methyl ethyl ketone.

23. (canceled)

24. (canceled)

25. The process as claimed in claim 14, wherein said ester of nitrile herbicide (Formula I) is selected from the group consisting of bromoxynil pentanoate, bromoxynil hexanoate, bromoxynil heptanoate, bromoxynil octanoate, bromoxynil nonanoate and bromoxynil decanoate.

26. A herbidical composition comprising at least one nitrile herbicide (Formula II) as claimed in claim 1, at least one ester of nitrile herbicide (Formula 1) as claimed in claim 1, and salts thereof.

27. The composition as claimed in claim 19, comprises bromoxynil heptonoate and bromoxynil octanoate in a mass ratio in the range of 1:99 to 99:1.

28. (canceled)
